(19) United States
(12) Patent Application Publication
(10) Pub. No.: US 2026/0226515 A1
(43) Pub. Date: Aug. 6, 2026

(54) PRODUCTION OF OMEGA-HYDROXY FATTY ACIDS AND THEIR DERIVATIVES USING ENGINEERED CELLS

(71) Applicant: NUTRITION & BIOSCIEN USA 4, INC., Rochester, NY (US)

(72) Inventors: ZACHARY BAER, Wilmington, DE (US); Michael W. BOSTICK, Wilmington, DE (US); Zhongqiang CHEN, Wilmington, DE (US); Xiaochun FAN, West Chester, PA (US); Min QI, Hockessin, DE (US); Yehong Jamie WANG, Wilmington, DE (US); Zhixiong XUE, Wilmington, DE (US); Quinn Qun zhu, West Chester, PA (US)

(21) Appl. No.: 19/146,092

(22) PCT Filed: Jan. 9, 2024
(86) PCT No.: PCT/EP2024/050391

§ 371 Date: Jul. 7, 2025

Related U.S. Application Data
(60) Provisional application No. 63/438,013, filed on Jan. 10, 2023.

(51) Int. Cl. C12P 7/6409; C12N 1/14; C12N 9/04; C12N 9/10; C12R 1/645

(52) U.S. Cl. CPC C12P 7/6409; C12N 1/14; C12N 9/0006; C12N 9/1029; C12R 2001/645; C12Y 101/03013; C12Y 203/0102; C12Y 203/01158

(57) ABSTRACT

Methods for producing macrocyclic molecules, particularly omega-hydroxy fatty acids, are described. The methods involve the use of engineered cells having modified biochemical pathways that allow them to efficient produce omega-hydroxy fatty acids from C16 precursors. The resulting omega-hydroxy fatty acids are useful for the production of perfumes and other scented consumer products.

### TECHNICAL FIELD

Methods for producing macrocyclic molecules, particularly omega-hydroxy fatty acids, are described. The methods involve the use of engineered cells having modified biochemical pathways that allow them to efficient produce omega-hydroxy fatty acids from C16 precursors. The resulting omega-hydroxy fatty acids are useful for the production of perfumes and other scented consumer products.

### BACKGROUND

Ambrettolide, iso-ambrettolide(s), hexadecanolide and innumerable related molecules are macrocyclic molecules that have exceptional diffusion properties and fine musk characteristics. These fragrance molecules can be produced from C16 omega-hydroxylated (ω-hydroxy) fatty acids. Currently, most ω-hydroxy fatty acid derivatives are made chemically from petroleum-based starting materials or through the bioconversion of paraffin. The required chemical methods for producing these compounds involve the use of hazardous organic reagents, are energy intensive and are environmentally costly.

The need exits for safer and more environmentally friendly methods for producing macrocyclic fragrance molecules that ideally use renewable feedstocks and are more cost effective than conventional chemical methods.

### SUMMARY

Described are methods for producing macrocyclic molecules, particularly omega-hydroxy (ω-hydroxy) fatty acids using engineered cells with modified biochemical pathways. The resulting (ω-hydroxy) fatty acids are useful for producing perfumes and other scented consumer products. Aspects and embodiments of the methods are described in the following, independently numbered paragraphs.
1. In one aspect, genetically engineered cells with reduced fatty alcohol oxidase activity and reduced fatty acid β-oxidation compared to otherwise identical parental cells are provided, wherein the genetically engineered cells are engineered to reduce or eliminate expression or activity of peroxisomal β-oxidation multifunctional enzyme type 2 (MFE2) and to reduce or eliminate expression or activity of endogenous fatty alcohol oxidase.
2. In some embodiments of the genetically engineered cells of paragraph 1, the fatty alcohol oxidase is fatty alcohol oxidase 1 (FAO1).
3. In some embodiments of the genetically engineered cells of paragraph 1 or 2, the cells are further engineered to reduce or eliminate the expression or activity of one or more diacylglycerol acyltransferases.
4. In some embodiments of the genetically engineered cells of paragraph 3, the diacylglycerol acyltransferase is acyl-CoA:diacylglycerol acyltransferase 1 (DGAT1) and/or acyl-CoA:diacylglycerol acyltransferase 2 (DGAT2) or phospholipid:diacylglycerol acyltransferase (PDAT) is functionally and/or structurally similar to proteins or homologs or having at least 60% degree of amino sequences described.
5. In some embodiments of the genetically engineered cells of paragraphs 1-4, the cells are further engineered to reduce or eliminate the expression or activity of a peroxisomal membrane protein.
6. In some embodiments of the genetically engineered cells of any of paragraphs 1-5, the cells are further engineered to increase the expression or activity of one or more endogenous, or over-express one or more exogenous cytochrome P450 proteins.
7. In some embodiments of the genetically engineered cells of paragraphs 1-6, the strain is a Yarrowia strain.
8. In some embodiments of the genetically engineered cells of paragraph 7, the strain is a Yarrowia lipolytica strain.
9. In another aspect, a method for the production of ω-hydroxy fatty acids from a hydrophobic substrate is provided, comprising: (a) providing genetically engineered cells according to any of paragraphs 1-7; (b) cultivating the strain in a suitable cultivation medium; and (c) contacting the strain with the hydrophobic substrate to form one or more ω-hydroxy fatty acids.
10. In some embodiments, the method of paragraph 9, further comprises (d) isolating the one or more β-hydroxy fatty acids.

These and other aspect and embodiments of the variant molecules and methods are described below, with reference to any incorporated Drawings.

### BRIEF DESCRIPTION OF THE DRAWINGS

FIG. 1 illustrates the β- and ω-oxidation pathways in Yarrowia.

FIG. 2 shows a physical and functional map of plasmid pYRH213.

FIG. 3 shows a physical and functional map of the AscI/SphI DNA fragment of plasmid pYRH213.

FIG. 4 illustrates the production of iso-ambrettolide from palmitic acid.

FIG. 5 illustrates the production of ambrettolide from palmitic acid.

### DETAILED DESCRIPTION

### 1. Definitions and Abbreviations

Prior to describing the variants and methods in detail, the following terms are defined. Unless defined otherwise, all technical and scientific terms used herein have the same meaning as commonly understood by one of ordinary skill in the art. The singular forms “a,” “an,” and “the” include plural referents unless the context clearly dictates otherwise. The present document is organized into a number of sections for ease of reading; however, the reader will appreciate that statements made in one section may apply to other sections. In this manner, the headings used for different sections of the disclosure should not be construed as limiting.

### 1.1. Definitions

As used herein, the term “ambrox” refers to (3aR,5aS,9aS,9bR)-dodecahydro-3a,6,6,9a-tetramethylnaphtho [2,1-b]furan), which is known commercially as AMBROX (Firmenich), Ambroxan (Henkel) AMBROFIX® (Givaudan), AMBERLYN® (Quest), CETALOX® Laevo (Firmenich), AMBERMOR® (International Flavors and Fragrances, and AROMOR® and/or norambrenolide Ether (Pacific). The desirable sensory benefits of ambrox come from the (−) stereoisomer rather than the (+) enantiomer. The odor of the (−) stereoisomer is described as musk-like, woody, warm or ambery whereas the (+) enantiomer has a relatively weak odor note.

As used herein, the term “activity” means the ability of an enzyme to react with a substrate to provide a target product. The activity can be determined in what is known as an activity test via the increase of the target product, the decrease of the substrate (or starting materials) or via a combination of these parameters as a function of time.

As used herein, the term “nucleic acid molecule,” refers to polynucleotides of the disclosure which can be DNA, cDNA, genomic DNA, synthetic DNA, or RNA, and can be double-stranded or single-stranded, the sense and/or an antisense strand.

As used, herein, an “expression vector” includes a recombinant nucleic acid molecule encoding polypeptides, including necessary regulatory regions suitable for expressing the polypeptides.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, “functionally and/or structurally similar proteins” are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 60%% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence described or referenced in the present patent application.

Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA.

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter:amino acid coding region:terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” may be used interchangeably and refer to genes, proteins or strains found in nature, or that are not intentionally modified for the advantage of the presently described cells/strains.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation,” “genetic alteration”, “genetic engineering”, and similar terms are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, altering signal transduction to turn on gene transcription or translation inside the cell, and combinations, thereof.

As used herein, the term “transformed” refers to the introduction of an exogenous or heterologous DNA into a cell. The transforming DNA may or may not be integrated, i.e., covalently linked into the genome of the cell.

As used herein, the phrase “engineered cells,” “modified yeast cells” or similar phrases, refer to cells that include genetic modifications and characteristics described herein.

Engineered/modified yeast do not include naturally occurring yeast.

### 1.2 Abbreviations and Acronyms

The following abbreviations/acronyms have the following meanings unless otherwise specified:
° C. degrees Centigrade
C16 pertaining to 16 carbon atoms in length
dH₂O or DI deionized water
FA fatty acid
g or gm grams
GC gas chromatography
hr(s) hour/hours
kg kilograms
M molar
mg milligrams
min(s) minute/minutes
mL and ml milliliters
mm millimeters
mM millimolar
sec seconds
sp. species
U units
v/v volume/volume
w/v weight/volume
w/w weight/weight
wt % weight percent

### 2. Introduction: Methods for Producing C-16 Derived Molecules in Cells

The present compositions and methods relate to genetically engineered cells that produce fragrance precursor molecules from C16 saturated, or unsaturated fatty acids. These molecules can be used to produce, e.g., hexadecanolide, ambrettolide, iso-ambrettolide and/or other fragrance molecules. Use of engineered cells will allow for the use of cost-effective fermentation processes to produce these precursor molecules.

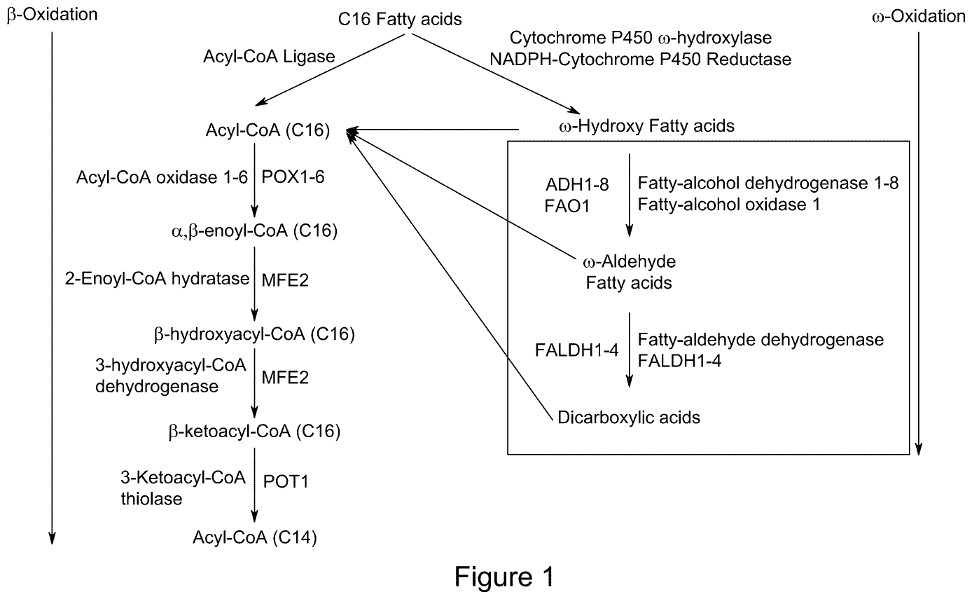

The theory behind the compositions and methods is that cells with pathways capable of producing fragrance precursor molecules need to be engineered to reduce or eliminate lipid storage, reduce or eliminate β-oxidation and reduce or eliminate the conversion of ω-hydroxy fatty acids to diacids, in order to effectively and efficiently produce ω-hydroxy fatty acids. Enzymatic reactions in the underlying pathway are shown in FIG. 1.

### 3. Reducing β-Oxidation in Cells to Increase the Production of

Many cells produce a peroxisomal β-oxidation multifunctional enzyme (MFE), specifically MFE type 2 (MFE2) involved in the fatty acid β-oxidation pathway, which is part of normal lipid metabolism. An aspect of the present methods involves the elimination or reduction of such activity to reduce fatty acid β-oxidation and increase the production of ω-hydroxy fatty acids (FIG. 1). Such engineered cells are capable of producing significantly more C16 ω-hydroxy fatty acids, compared to otherwise identical parental cells. As illustrated, Yarrowia, expresses six acyl-coA oxidases (POX1 to POX6) that carry out the first step of β-oxidation, these enzymes are substrate specific. MFE2 is the only enzyme responsible for the second and third step of the β-oxidation.

The elimination or reduction of MFE2 activity can be combined with elimination or reduction of fatty alcohol oxidase 1 (FAO1) activity, as described in U.S. Pat. No. 10,093,950, which reduces the conversion of ω-hydroxy fatty acids to dicarboxylic acids, resulting in even further increased production of ω-hydroxy fatty acids, which are precursors for more valuable fragrance molecules. Alternatively, elimination or reduction of MFE2 activity is performed without elimination or reduction of fatty alcohol oxidase 1 (FAO1) activity.

### 4. Mutations for Reducing Lipid Biosynthesis and Accumulation

The peroxisomal membrane protein (PMP) Pex3, and its interaction partner, cytosolic Pex19, have been implicated in peroxisomal membrane biogenesis. Their mode of operation is unclear. Pex3 can also recruit other proteins to the peroxisomal membrane to affect autophagy and organelle retention. Reducing or eliminating the activity of Pex3, or a functionally and/or structurally similar proteins, resulted in the elimination of functional peroxisomes. Reducing or eliminating the activity of Pex5 and Pex10 is expected to have similar results.

Eukaryotes commonly express up to three distinct classes of diacylglycerol (DAG) acyltransferases, namely acyl-CoA:diacylglycerol acyltransferase 1 (DGAT1), acyl-CoA:diacylglycerol acyltransferase 2 (DGAT2), and phospholipid:diacylglycerol acyltransferase (PDAT). Yarrowia contains at least one homologue of each molecule. These genes, or genes that encode functionally and/or structurally similar proteins, can be disrupted to significantly reduce oil biosynthesis and accumulation compared to other wise identical parental cells.

### 5. Further Increased Production of C16:0 ω-Hydroxy Fatty Acids

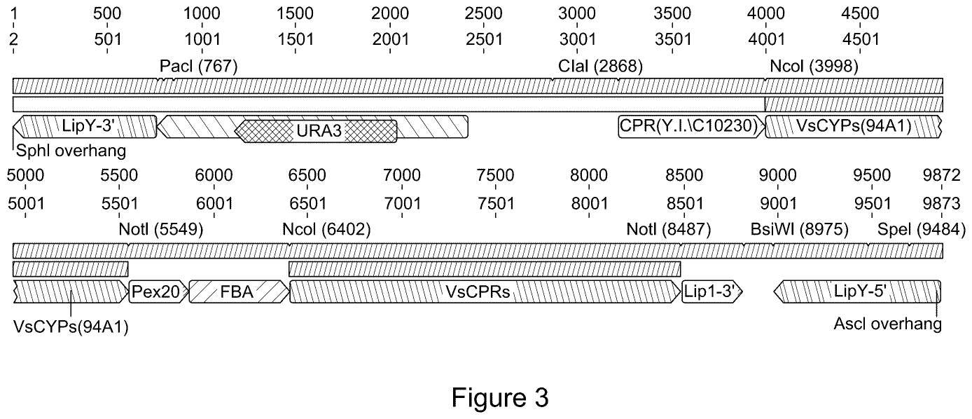

A further increase in the production of ω-hydroxy fatty acids can be obtained by over-expressing endogenous cytochrome P450 (CYP) enzyme, or expressing more efficient exogenous CYP enzymes in cells, channeling C16 fatty acids toward conversion to ω-hydroxy fatty acids (FIG. 1). For this purpose, two expression cassettes were introduced into cells, which are illustrated in FIG. 3. The cassettes introduced slightly different codon-optimized sequences encoding CYP enzymes derived from Vicia sativa (i.e., VsCYP94A1; GenBank ACC. No AAD10204; and VsCPR; GenBank Acc. No. Z26252.

### 6. Cells for Producing C16:1(Δ9) ω-Hydroxy Fatty Acids

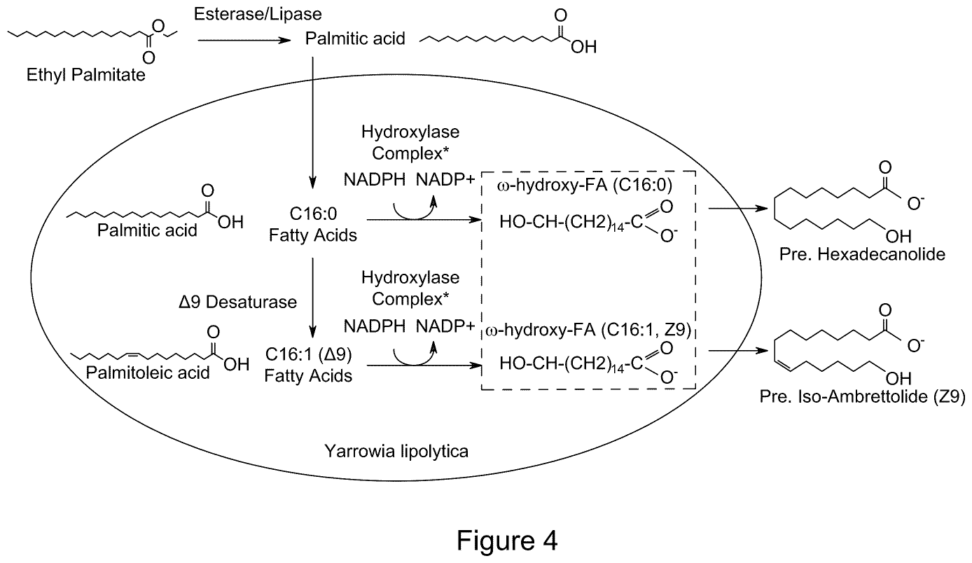

Cells with reduced or eliminated lipid storage, β-oxidation and conversion of ω-hydroxy fatty acids to diacids can be used to effectively produce C16:1(Δ9) ω-fatty acids, such as those produced from plant oil, plant oil-derived fatty acids, or fatty acid esters by expression, or over-expression, of the ω-hydroxylase complex with Δ9 desaturase (stearoyl-CoA 9-desaturase) that can convert palmitic acid to palmitoleic acid (FIG. 4).

Useful Δ9 desaturases are those from yeast and plants. Especially useful Δ9 desaturases are from Yarrowia (GenBank Acc. No. YALI0C05951), Saccharomyces (GenBank Acc. No. NP_011460) or Δ9 desaturases with 80% amino acid sequence identity to either of these enzymes.

### 7. Cells for Producing C16:1(Δ7) ω-Hydroxy Fatty Acids

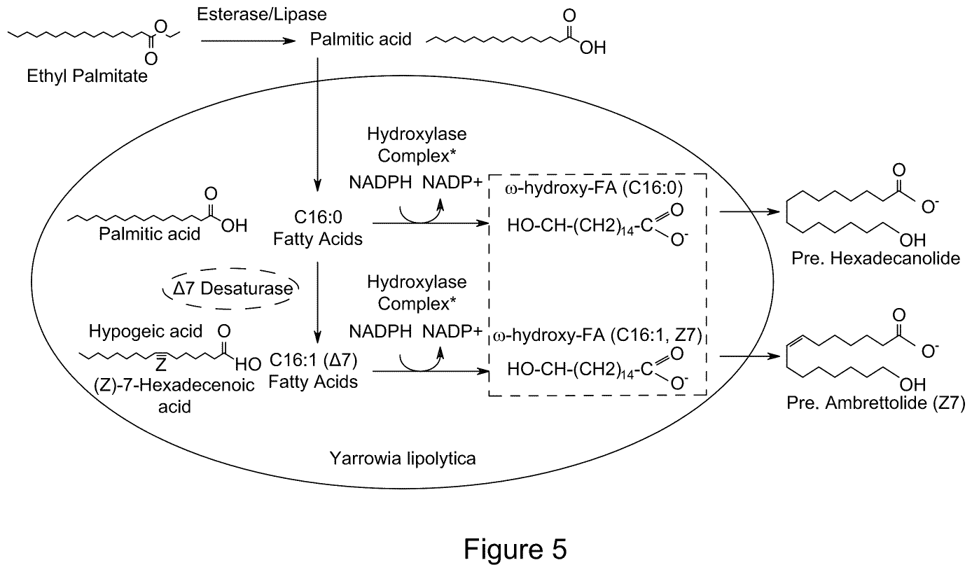

Cells with reduced or eliminated lipid storage, β-oxidation and conversion of ω-hydroxy fatty acids to diacids can also be used to effectively produce C16:1(Δ7) omega-fatty acids from plant oil, plant oil-derived fatty acids, or fatty acid esters by co-expression of the omega hydroxylase complex with Δ7 desaturase, namely, palmitoyl-monogalactosyldiacylglycerol desaturase, which can convert palmitic acid to (Z)-7-hexadecenoic acid (FIG. 5). Useful Δ7 desaturases are those that can be effectively expressed and are functional. Especially useful enzymes are those from plants and/or algae.

As with Δ7 desaturases, monogalactosyldiacylglycerol (MGDG) synthase exists in the chloroplasts of plants and eukaryotic green algae. The enzyme catalyzes the formation of MGDG, which is a major structural and functional lipid on chloroplasts. Useful MGDG synthase for this application would be those that can be effectively expressed and are functional. Especially useful enzymes are those from plants and/or algae that produce C16:1 (Δ7) fatty acids.

### 8. Additional Mutations to Control Cell Growth

The mating-type (MATa) locus of cells includes genes responsible for inducing sporulation in a diploid B/B cells, repressing the mating capacity of the cells. Genes in the MATa locus can be disrupted to prevent sporulation.

URA3 is the conventional selection marker used in a yeast cells because it enables auxotrophic transformant screening as well as counterselection potential for marker removal, via 5′-FOA resistance.

### 9. Suitable Microorganism for Pathway Engineering

Any fungal cells can be used according to the present methods, especially oleaginous yeast, or yeast that are engineered to be oleaginous yeast. Such yeast include, but are not limited to, Yarrowia, Candida and Saccharomyces.

### 10. Recovery or Fragrance Molecules and Precursor Molecules from Cells

ω-hydroxy fatty acids and their derivatives produced by the present methods may be collected, e.g., by steam extraction/distillation or organic solvent extraction using a non-water miscible solvent (to separate the reaction products and unreacted substrate from the biocatalyst which stays in the aqueous phase) followed by subsequent evaporation of the solvent to obtain a crude reaction product as determined by gas chromatographic (GC) analysis.

The molecules may be further selectively crystallized to remove unreacted substrate from the final product. In some embodiments, the isolated crystalline material contains only the desired enantiomers. In other embodiments, the isolated crystalline material contains the other isomers, wherein said isomers are present only in olfactory acceptable amounts.

Examples of suitable water miscible and non-water miscible organic solvents suitable for use in the extraction and/or selective crystallization of ω-hydroxy fatty acids and their derivatives include, but are not limited to, aliphatic hydrocarbons, preferably those having 5-8 carbon atoms, such as pentane, cyclopentane, hexane, cyclohexane, heptane, octane or cyclooctane; halogenated aliphatic hydrocarbons, preferably those having one or two carbon atoms, such as dichloromethane, chloroform, carbon tetrachloride, dichloroethane or tetrachloroethane; aromatic hydrocarbons, such as benzene, toluene, the xylenes, chlorobenzene or dichlorobenzene; aliphatic acyclic and cyclic ethers or alcohols, preferably those having 4-8 carbon atoms, such as ethanol, isopropanol, diethyl ether, methyl tert-butyl ether, ethyl tert-butyl ether, dipropyl ether, diisopropyl ether, dibutyl ether, tetrahydrofuran; or esters such as ethyl acetate or n-butyl acetate or ketones such as methyl isobutyl ketone or dioxane or mixtures of these. The solvents that are especially preferably used are the above-mentioned heptane, methyl tert-butyl ether (also known as MTBE, tertiary butyl methyl ether and iBME), diisopropyl ether, tetrahydrofuran, ethyl acetate and/or mixtures thereof. Preferably, a water miscible solvent such as ethanol is used for the extraction of (−)-sclareolide from the solid phase of the reaction mixture. The use of ethanol is advantageous because it is easy to handle, it is non-toxic and it is environmentally friendly.

### 11. Applications for Cell-Derived ω-Hydroxy Fatty Acid-Derived Molecules

Various applications for ω-hydroxy fatty acids and their derivatives include, but are not limited to, non-enzymatically producing ambrettolide and/or ambrettolide-like molecules. and other fragrance molecules for use in a fine fragrance or a consumer product such as fabric care, toiletries, beauty care and cleaning products.

The following non-limiting examples are provided to further illustrate the present methods.

### EXAMPLES

### Example 1: Generation of Cells for Production of C16 ω-Hydroxy Fatty Acids

A parental strain of Yarrowia lipolytica (ATCC No. 20362) having the biochemical pathway illustrated in FIG. 1 was selected for genetic manipulation to engineer a strain that could produce high amounts of ω-hydroxy fatty acids.

To this end, the parental strain of Yarrowia was engineered to reduce or eliminate lipid storage, β-oxidation and the conversion of ω-hydroxy fatty acids to diacids. As summarized Table 1 two new modified strains with unique genotypes were generated from the parental wild-type strain ATCC No. 20362. Strain AH007 had a wild-type MFE2 gene. Strain AH085 had a deleted MFE2 gene.

TABLE 1
Yarrowia strains for production of ω-hydroxy fatty acids

| Strain | Genotype | Reference |
|---|---|---|
| ATCC No. | MATA | ATCC |
| 20362 | | |
| Y2224 | ura3⁻, MATA | U.S. Pat. No. |
| | | 10,626,424B2 |
| D0003 | dgat1⁻, dgat2⁻, ura3⁻, MATA | H. et al. |
| D0004 | dgat1⁻, dgat2⁻, pex3⁻, ura3⁻, | U.S. Pat. No. |
| | MATA | 10,626,424B2 |
| AH007 | dgat1⁻, dgat2⁻, pex3⁻, fao1⁻, | Instant disclosure |
| | ura3⁻, MATA | |
| AH085 | dgat1⁻, dgat2⁻, pex3⁻, fao1⁻, | Instant disclosure |
| | mfe2⁻, ura3⁻, MATA | |
| H. et al. | | |

### Example 2: Generation of Cells for Further Increased Production of C16:0 ω-Hydroxy Fatty Acids

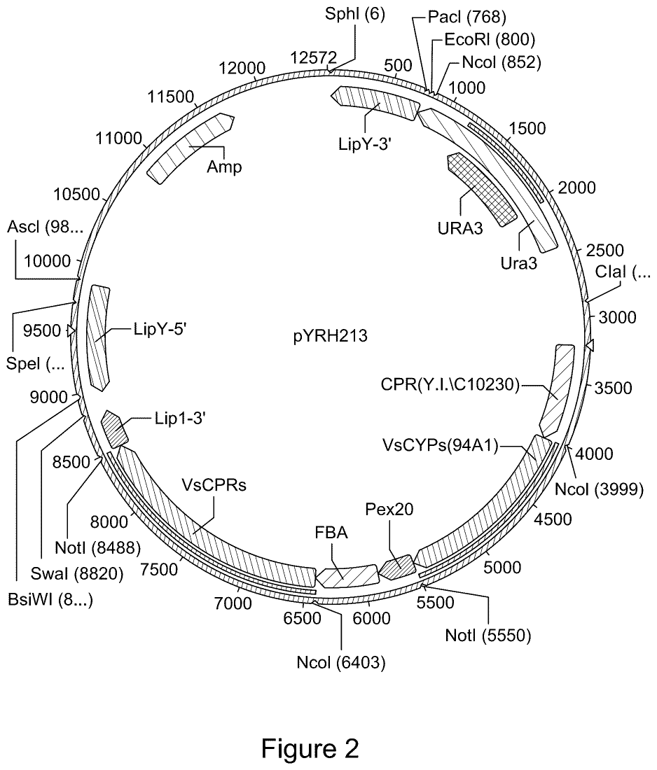

For construction of a Yarrowia strain to further over-produce ω-hydroxy fatty acids, cells of strain AH085 from Example 1 were transformed with an artificial plasmid (FIG. 2) containing two expression cassettes FIG. 3 for over-expressing slightly different codon-optimized sequences encoding CYP enzymes derived from Vicia sativa (i.e., VsCYP94A1; GenBank ACC. No AAD10204; SEQ ID NO: 14 encoding SEQ ID NO: 13 and VsCPR; GenBank Acc. No. Z26252; SEQ ID NO: 16 encoding SEQ ID NO: 15).

Restriction endonuclease sites were introduced upstream of the translation initiation codons and downstream of the stop codons of each codon-optimized sequence to enable the excision of polynucleotides including the VsCYP or VsCPR-coding sequences for transformation into AH085 cells.

Resulting transformant cells were plated onto minimal media plates and incubated at 30° C. for 2 days. Individual colonies from each transformation were re-streaked onto MM plates. 24 strains were directly analyzed for ω-hydroxy fatty acid production using block assays. Specifically, individual colonies were re-streaked onto MM plates, and then inoculated into liquid Y2P1D2-B media (20 g/L yeast extract, 10 g/L peptone, 20 g/L glucose, 16.37 g/L K₂HPO₄, 0.82 g/L KH₂PO₄ and 0.2 ml/L; trace metals (100×): 1 ml/L thiamine-HCl (75 mg/ml), 0.5 ml/L 1 M MgSO₄—·7H₂0, 1 ml/L and 50 mg/ml kanamycin; Trace Metals Recipe (100×): 10.0 g/L citric acid, 1.5 g/L CaCl₂·2H₂O, 10.0 g/L FeSO₄·7H₂O, 0.39 g/L ZnSO₄·7H₂O, 0.38 g/L CuSO₄·5H₂O, 0.20 g/L CoCl₂·6H₂O and 0.30 g/L MnCl₂·4H₂O) in 24-well blocks, which were then shaken at 30° C. and 375 rpm for 20 hr. The cultures were adjusted to pH 8.0 with addition of 0.12 mL of 1 M NaHCO₃, after which ethyl palmitate was added directly to the culture media to a final concentration of 23 mg/mL. The cultures were then shaken for an additional 2 days at 375 rpm at 30° C., after which whole broth samples from each culture were subjected to ω-hydroxy fatty acid analysis according to known methods.

GC analyses demonstrated that most transformants from these two sets of transformations produced C16 ω-hydroxy fatty acid (data not shown). Both AH007 and AH085 produced only about 1 g/L C16 ω-hydroxy fatty acids. One strain, designated H021, derived from parent strain AH007, produced 8.1 g/L C16 ω-hydroxy fatty acids. Another strain, designated H088, derived from AH085, produced 10.1 g/L. These data demonstrated that mfe2Δ deletion resulted in ~25% increase of the levels of C16 ω-hydroxy fatty acids. Similarly, the amount of C16:0 diacid produced by strain H088 also increased >25% compared to the amount produced by strain H021

The fatty acid composition of whole broths obtained from strains H021 and H088 are summarized in Table 2, base3d on 3-day block assays

TABLE 2
Fatty Acid composition of the whole broth obtained from strains H021 and H088

| | Fatty acids (g/L) in whole broth | | | | | | | | | |
|---|---|---|---|---|---|---|---|---|---|---|
| | C16:0 | C16:1 | C18:1 | C18:2 | | | C16:0 | | | |
| | FA | (Δ9) | (Δ9) | (Δ9, 12) | C16:0 | C16:1 | Hydroxy | C18:0 | C18:1 | C18:2 |
| Strains | substrate | FA | FA | FA | Diacid | Diacid | FA | Diacid | Diacid | Diacid |
| H021 | 10.8 | 0.2 | 0.2 | 0.5 | 8.6 | 0.7 | 8.1 | 0.1 | 0.4 | 0.3 |
| H088 | 12.9 | 0.1 | 0.2 | 0.4 | 10.8 | 0.5 | 10.1 | 0.3 | 0.5 | 0.2 |

### Example 3: (Prophetic) Generation of Cells for Production of C16:1(Δ9) ω-Hydroxy Fatty Acids

Cells with reduced or eliminated lipid storage, β-oxidation and the conversion of ω-hydroxy fatty acids to diacids can be used to effectively produce C16:1(Δ9) ω-fatty acids, such as those produced from plant oil, plant oil-derived fatty acids, or fatty acid esters by co-expression of the ω-hydroxylase complex (Example 2) along with Δ9 desaturase (stearoyl-CoA 9-desaturase) that can convert palmitic acid to palmitoleic acid (FIG. 4).

### Example 4: (Prophetic) Generation of Cells for Production of C16:1(Δ7) ω-Hydroxy Fatty Acid

Cells with reduced or eliminated lipid storage, β-oxidation and the conversion of ω-hydroxy fatty acids to diacids can be used to effectively produce C16:1(Δ7) ω-fatty acids, such as those produced from plant oil, plant oil-derived fatty acids, or fatty acid esters by co-expression of the ω-hydroxylase complex (Example 2) along with Δ7 desaturase, namely palmitoyl-monogalactosyldiacylglycerol desaturase, that can convert palmitic acid to (Z)-7-hexadecenoic acid (FIG. 5), which can convert palmitic acid to palmitoleic acid.

1. Genetically engineered cells with reduced fatty alcohol oxidase activity and reduced fatty acid β-oxidation compared to otherwise identical parental cells, wherein the genetically engineered cells are engineered to reduce or eliminate expression or activity of peroxisomal β-oxidation multifunctional enzyme type 2 (MFE2) and to reduce or eliminate expression or activity of endogenous fatty alcohol oxidase.

2. The genetically engineered cells of claim 1, wherein the fatty alcohol oxidase is fatty alcohol oxidase 1 (FAO1).

3. The genetically engineered cells of claim 1, wherein the cells are further engineered to reduce or eliminate the expression or activity of one or more diacylglycerol acyltransferases.

4. The genetically engineered cells of claim 3, wherein the diacylglycerol acyltransferase is acyl-CoA:diacylglycerol acyltransferase 1 (DGAT1) and/or acyl-CoA:diacylglycerol acyltransferase 2 (DGAT2) or phospholipid:diacylglycerol acyltransferase (PDAT) is functionally and/or structurally similar to proteins or homologs or having at least 60% degree of amino sequences described.

5. The genetically engineered cells of claim 1, wherein the cells are further engineered to reduce or eliminate the expression or activity of a peroxisomal membrane protein.

6. The genetically engineered cells of claim 1, wherein the cells are further engineered to increase the expression or activity of one or more endogenous, or over-express one or more exogenous cytochrome P450 proteins.

7. The genetically engineered cells of claim 1, wherein the strain is a Yarrowia strain.

8. The genetically engineered cells of claim 7, wherein the strain is a Yarrowia lipolytica strain.

9. A method for the production of ω-hydroxy fatty acids from a hydrophobic substrate, comprising: (a) providing genetically engineered cells according to claim 1; (b) cultivating the strain in a suitable cultivation medium; and (c) contacting the strain with the hydrophobic substrate to form one or more ω-hydroxy fatty acids.

10. The method of claim 9, further comprising (d) isolating the one or more β-hydroxy fatty acids.
